(19) United States
(12) Patent Application Publication
(10) Pub. No.: US 2020/0054560 A1
(43) Pub. Date: Feb. 20, 2020

(54) PALBOCICLIB COMPOSITIONS AND METHODS THEREOF

(71) Applicant: Alnova Pharmaceuticals, Ltd., Shanghai (CN)

(72) Inventors: Xini Zhang, Green Brook, NJ (US); Pankaj Laxmikant Ingole, Natepute (IN); Zhigang Xiong, (US); Qian Lu, Shanghai (CN)

(73) Assignee: Alnova Pharmaceuticals, Ltd., Shanghai (CN)

(21) Appl. No.: 16/606,592

(22) PCT Filed: Apr. 21, 2017
(86) PCT No.: PCT/CN2017/081425

§ 371 Date: Oct. 18, 2019

(51) Int. Cl. A61K 9/14; A61K 9/16; A61K 9/48; A61K 31/519

(52) U.S. Cl. CPC A61K 9/146; A61K 31/519; A61K 9/4866; A61K 9/1652

(57) ABSTRACT

Palbociclib compositions with improved solubility and bioavailability, methods of their preparation, and methods of treating cancers using the compositions are disclosed.

### FIELD OF THE INVENTION

The invention relates to Palbociclib compositions with improved solubility and increased bioavailability, methods for preparation, and method of treatment for cancer.

### BACKGROUND OF THE INVENTION

The compound 6-acetyl-8-cyclopentyl-5-methil-2-(5-piperazin-1-yl-pyridin-2-ylamino)-8H-pyrido [2, 3-d] pyridin-7-one, apparently has the following chemical structure:

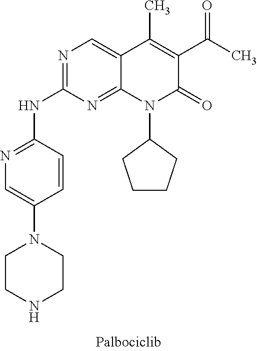 [SMILES: CC(=O)c1c(C)c2cnc(Nc3ccc(N4CCNCC4)cn3)nc2n(C2CCCC2)c1=O]

Palbociclib is reported to be a yellow to orange crystalline powder that is classified as BCS Class II Compound based on the Biopharmaceutics Classification System. It is slightly soluble in dimethyl sulfoxide and N,N-dimethylformamide, very slightly soluble in methanol and water.

Palbociclib is a kinase inhibitor indicated for the treatment of hormone receptor (HR)-positive, human epidermal growth factor receptor 2 (HER2)-negative advanced or metastatic breast cancer in combination with: Letrozole as initial endocrine based therapy in postmenopausal women, or Fulvestrant in women with disease progression following endocrine therapy. Palbociclib sold under brand name IBRANCE®, which is marketed by Pfizer. IBRANCE® is available as capsule for oral administration containing supposedly 125 mg, 100 mg and 75 mg of Palbociclib. The Inactive ingredients of IBRANCE are reported to be microcrystalline cellulose, lactose monohydrate, sodium starch glycolate, colloidal silicon dioxide, magnesium stearate, and hard gelatin capsule shells. The light orange, light orange/caramel and caramel opaque capsule shells contain gelatin, red iron oxide, yellow iron oxide, and titanium dioxide; and the printing ink contains shellac, titanium dioxide, ammonium hydroxide, propylene glycol and simethicone.

Palbociclib is reported to be very slightly soluble in water. When a solid dosage form of Palbociclib is taken orally, the drug must dissolve in aqueous gastrointestinal fluid in, e.g., the patient's stomach before it can exert a therapeutic effect. At or below pH 4, Palbociclib behaves as a high-solubility compound. Above pH 4, the solubility of the drug substance reduces significantly. A recurring problem with compressed solid oral dosage forms, such as tablets, capsules and caplets (i.e. capsules-shaped tablets) is that the rate of dissolution of the drug limits its biological availability.

Methods for improving dissolution by reducing particle size have been described in the past for water-insoluble drugs other than Palbociclib. However, particle size reduction is not always effective enough for increasing the dissolution rate of drug to a certain required value. Many water-insoluble drugs have a strong tendency to agglomerate during the dosage form manufacturing process into larger particles with an overall decrease in effective surface area. Remington: the Science and Practice of Pharmacy. 20th ed. 656,657 (A. R. Gennaro Ed., Lippincott Williams & Wilkins: Philadephia 2000), incorporated by reference herein, contains a more thorough discussion of the concept of “effective surface area” and the effect of particle size on dissolution. A drug that has ostensibly been milled to a fine particle size will sometimes display dissolution characteristics of a larger particle due to agglomeration or similar effect.

There is a need in the art for Palbociclib compositions with improved solubility and increased bioavailability.

### BRIEF SUMMARY OF THE INVENTION

The invention encompasses Palbociclib composition with improved solubility and increased bioavailability, method for preparation and method for treatment of cancer.

In one aspect the invention encompasses a Palbociclib composition comprising Palbociclib co-milled with at least one hydrophilic excipient. In another aspect, the invention encompasses a method for preparing a Palbociclib composition comprising co-milling Palbociclib and at least one hydrophilic excipient to form the Palbociclib composition.

The invention also encompasses Palbociclib composition prepared by a method of the invention. The invention further encompasses a method for treatment of breast cancer.

### DETAILED DESCRIPTION OF THE INVENTION

The invention encompasses Palbociclib composition with improved solubility and increased bioavailability, methods of their preparation, and methods of treatment using same.

The method for preparing Palbociclib free base solution comprises dissolving Palbociclib dihydrochloride in water to form a solution, adding the solution dropwise to a mixture of ammonium hydroxide (15%), methanol and dichloromethane to form an upper layer and a lower layer, separating layers, to collect the lower layer; and adding at least one of hydrophilic excipient to the collected lower layer.

The method for preparing the co-milled Palbociclib with at least one of hydrophilic excipients includes addition of hydrophilic excipient with organic solvent into the solution of Palbociclib in solvent (i.e. without isolated Palbociclib) to form dispersion, partially removing solvent, then drying stage followed by vacuum drying, dried blend of Palbociclib with at least one of hydrophilic excipient milled through using at least one of a jet mill, rolling mill, hammer mill, centrifugal-impact mill and sieve, pebble mill, cutter mill, runner mill or mortor and pestle.

It has been discovered that the solubility of Palbociclib is increased by addition of hydrophilic excipient, such as a saccharide or polysaccharides or disaccharide, e.g. starch, lactose to a composition containing milled Palbociclib.

In one aspect, the invention encompasses a Palbociclib composition comprising Palbociclib co-milled with at least one of hydrophilic excipient. The hydrophilic excipient may include a saccharide, polysaccharides, and/or disaccharides, (for example, starch, pregelatinized starch, mannitol, or sorbitol, lactose, or the like) calcium carbonate, cellulose, sorbitol, povidone, silicic acid, beta cyclodextrin, and/or polyethylene glycol.

In a preferred embodiment, the pharmaceutical composition of the invention has a dissolution rate of more than 85% within 30 minutes at pH 1.2.

Co-milling can be carried out using conventional milling processes, which include jet milling, rolling melling, hammer milling, centrifugal-impact milling and sieving, pebble milling, cutter milling, or use of a mortor and pestle. The co-milled Palbociclib may have a particle distribution, d(10) of about 50 μm or less, d(50) of about 200 μm or less, d(90) of about 400 μm or less, or d(10) of about 25 μm or less, d(50) of about 100 μm or less, d(90) of about 200 μm or less, or d(10) of about 15 μm or less, d(50) of about 50 μm or less, d(90) of about 100 μm or less, most preferably particle size is d(10) of 1 to 5 μm, d(50) 5 to 10 μm and d(90) less than 20 μm.

In one embodiment, the combination has a Palbociclib: Hydrophilic excipient weight ratio of about 1:10 to about 10:1, preferably from about 1:5 to about 5:1, more preferably about 1:3 to about 1:1 and even more preferably 1:2.

In one embodiment, the Palbociclib composition is in the form of granule. In another embodiment, a composition of the invention is a formulation further comprising one or more pharmaceutically acceptable excipient(s), in addition to the hydrophilic excipient. Preferably, about 85% or more of the Palbociclib composition comprising the additional pharmaceutical excipient is dissolved in 30 minutes. Optionally, the additional pharmaceutically acceptable excipient comprises at least one of binder, filler, disintegrant, or glidant, lubricant, and hard gelatin shell, more particularly, for example, Povidone, Microcrystalline cellulose, sodium starch glycolate, colloidal silicon dioxide, magnesium stearate, and hard gelatin capsule shells.

In another embodiment, the composition further comprises one or more dispersing agent, e.g., povidone.

In another aspect, the invention encompasses a method for preparing a Palbociclib composition comprising co-milling Palbociclib with at least one hydrophilic excipient to form the Palbociclib composition.

In one embodiment, the invention encompasses a method for preparing a Palbociclib composition comprising co-milling Palbociclib and at least one hydrophilic excipient, wherein about 40% or more, preferably about 40% to about 70%, more preferably about 50% or more, and even more preferably about 60% or more, of the Palbociclib composition.

In another embodiment, the composition further comprises an organic solvent to prepare Palbociclib dispersion. Organic solvent can be selected from the group consisting of methanol, ethanol, isopropyl alcohol, ethyl acetone, dichloromethane, tetrahydrofuran, and their mixtures.

A Palbociclib composition may be prepared by methods known in the art, such as dry granulation, wet granulation, direct compression. Preferably, the composition is prepared by wet granulation or dry granulation. The wet granulation mixture contains a dispersing agent, which preferably comprises with or without binder addition, e.g., povidone. The co-milled Palbociclib and hydrophilic excipient may be combined with one or more pharmaceutically acceptable excipient, such as a binder, filler, disintegrant, glidant, and/or lubricant.

The method for preparing the Palbociclib composition may further comprising slugging the co-milled Palbociclib and Hydrophilic excipient to form slugs, and milling the slugs into a powder; or passing the co-milled Palbociclib and hydrophilic excipient through a screen to form granulates.

In another embodiment, the invention encompasses a method for preparing a Palbociclib composition comprising co-milling Palbociclib and stach/lactose to an average particle size of less than 20 μm; blended with a disintegrant such as sodium starch glycolate, croscarmellose sodium, etc. To prepare slug use co-milled Palbociclib with disintegrant blend; milling the slugs by using multi mill with 2.5 mm screen followed by sieving with 1.5 mm screen; sift all granules through 20 mesh screen, mixing with extragranular excipients followed by encapsulation into hard gelatin capsules or compressing into tablet form.

Palbociclib compositions of the present invention can contain inactive ingredients such as diluents, carriers, fillers, binder, disintegrants, glidants, lubricants.

Diluents increase the bulk of solid pharmaceutical composition and can make pharmaceutical dosage form containing the composition easier for the patient and care giver to handle. Diluent for solid composition includes, for example, microcrystalline cellulose, lactose, starch, pregelatinized starch, mannitol, and/or dibasic calcium phosphate, etc.

Carrier for use in the compositions may include, but are not limited to, pregelatinized starch, mannitol, sorbitol, lactose, calcium carbonate, cellulose, povidone, silicic acid, beta cyclodextrin, polyethylene glycol, and the like.

Binders help bind the active ingredient and other excipients together after compression. Binder for solid pharmaceutical compositions include, for example, carbomer (e.g. carbopol), carboxymethylcellulose sodium, ethyl cellulose, hydroxypropyl cellulose (e.g., Klucel), hydroxy propyl methyl cellulose (e.g., Methocel) and povidone (e.g., Kollidon, Plasdone)

Disintegrant can increase dissolution. Disintegrants include, for example, carboxymethylcellulose sodium (e.g. Ac-Di-Sol, Primellose), Sodium starch Glycolate, Collidal silicon dioxide, crospovidone (e.g. Kollidon, Polyplasone), microcrystalline cellulose, and starch, etc.

A lubricant can be added to the composition to reduce adhesion and ease release of the product from a punch or dye during encapsulation. Lubricants include, for example, magnesium stearate, hydrogenated castrol oil, mineral oil, polyethylene glycol, sodium steryl fumarate, and sodium lauryl sulfates.

Glidants can be added to improve the flowability of non-compacted composition and improve the accuracy of dosing. Excipients that can function as glidants include, for example, colloidla silicon dioxide, magnesium trisilicate, powdered celloulose, starch, and talc, etc.

Capsules can be filled with powder or granule composition of the invention.

As described above, the Palbociclib formulation of the invention can be prepared by wet granulation by using co-milled Palbociclib, and excipients in powder form are blended and then further mixed in the presence of liquid, typically water and/or alcohol or Hydroalcoholic, which causes the powders to clump up into granules. The granulation solution may or may not contain a dispersing agent such as povidone. The granulate so formed is optionally screened and/or milled, dried and then screened and/or milled through 30 mesh screen. The granulates can then encapsulated or other excipients can be added prior to encapsulation, such as glidant, lubricant, and/or disintegrant.

In another embodiment, the composition further comprises organic solvent for wet granulation process. Organic solvent is selected from the group of methanol, ethanol, isopropyl alcohol, and their mixtures.

A preferred dosage form is capsule. A composition can be prepared conventionally by dry granulation. For instance, the co-milled Palbociclib and other excipients can be compacted into slug then comminuted into compacted granules. The compressed granules can be filled into capsule.

As an alternative to dry granulation, a blended composition can be compressed directly into a compacted dosage form using direct compression techniques.

Alternatively, co-milled Palbociclib can be directly mixed with extragranular excipients and filled in hard gelation capsules.

A capsule filling of present invention can comprise any of the aforementioned blends and granulates that are described with reference to tabletting, except that they are not subjected to a final tabletting step.

The invention also encompasses Palbociclib compositions prepared by the methods of the invention. The invention also encompasses a method of treatment of breast cancer.

### EXAMPLES

The following examples serve to illustrate the compounds in this invention and the preparation process, but the examples should not be considered as limiting the scope of the invention.

### Example 1

Effect of particle size and addition of starch/lactose on dissolution rate of Palbociclib.

The method for preparing the co-milled Palbociclib with one of hydrophilic excipients includes the steps:

1. Addition of hydrophilic excipient with Organic solvent into a solution of Palbociclib free base in organic solvent (without isolated Palbociclib) to form dispersion.

2. Removing solvent, followed by vacuum drying, dried blend of Palbociclib with at least one of hydrophilic excipient.

3. Milled step (2) dried mixture through at least one of a jet mill, rolling mill, hammer mill, centrifugal-impact mill and sieve, pebble mill, cutter mill, runner mill, mortor and pestle.

4. The Palbociclib was milled to an estimated size of d(10) of 1 to 5 μm, d(50) 5 to 10 μm and d(90) less than 20 μm. The samples were prepared as follows:

1) 125 mg of milled Palbociclib d 90 (less than 20 μm),

2) 125 mg of non-milled Palbociclib d 90 (160 to 180 μm),

3) 125 mg of milled Palbociclib blended with 250 mg of pregelatinized starch (Starch 1500), and

4) 125 mg of non-milled Palbociclib blended with 250 mg of pregelatinized starch (Starch 1500),

5) Co-milled, 125 mg of Palbociclib with 250 mg of starch 1500.

The dissolution profiles of each sample were tested using following method as Table 1:

TABLE 1

| Apparatus | II (Paddle) with sinker | |
|---|---|---|
| Medium | 0.1N | HCl |
| Volume | 900 | ml |
| Temperature | 37° | C. |

| | |
|---|---|
| Speed | 50 |
| Sampling points | 10, 15, 20, 30 and 45 minutes |

The dissolution rate increased for the milled Palbociclib blended with Starch/Lactose.

### Example 2

Table 2 shows the preparation of Palbociclib free base solvent:

TABLE 2

| | Ingredient | mg/cap |
|---|---|---|
| Palbociclib free base | Palbociclib | 145.33 |
| solution in Methanol/ | dihydrochloride | |
| Dichloromethane | Water | 726.67 |
| | Ammonium hydroxide | 63.33 |
| | (15%) | |
| | Methanol | 1000.00 |
| | Dichloromethane | 5000.00 |

| | |
|---|---|
| Theoretical end weight of Palbociclib free base | Palbociclib solvent |
| | eq. to 125 mg of |
| | palbociclib free base. |

For Preparation of 150 Capsules:

Palbociclib dihydrochloride (21.80 g) was dissolved in water (109 ml). The above solution was added dropwise to the mixture of ammonium hydroxide (15%, 9.5 g), Methanol (150 g) and dichloromethane (745 g). Then the layers were separated. Discarded the upper layer and collected the lower layer for further process.

### Example 3 to 5

Table 3 shows the formulation of Palbociclib by wet granulation process:

TABLE 3

| Examples | | 3 | | 4 | | 5 | |
|---|---|---|---|---|---|---|---|
| | Ingredient | mg/cap | % | mg/cap | % | mg/cap | % |
| Part I | Palbociclib free base | Eq. to | 31.25 | Eq. to | 31.25 | Eq. to | 31.25 |
| (Dispersion, | solution | 125.00@ | | 125.00@ | | 125.00@ | |
| drying, | (in Methanol/ | | | | | | |
| Co-Milling) | Dichloromethane) | | | | | | |
| | from example 2 | | | | | | |
| | Starch 1500 | 250.00 | 62.50 | NA | NA | NA | NA |
| | Lactose Monohydrate | NA | NA | 250.00 | 62.50 | 250.00 | 62.50 |
| Part II (Binder | Povidone | 5.00 | 1.25 | 5.00 | 1.25 | NA | NA |
| solution) | Ethanol 97% * | 100 ml | NA | 100 ml | NA | NA | NA |
| Extra | Microcrystalline | 15.00 | 3.75 | 15.00 | 3.75 | 20.00 | 5.00 |
| granular | cellulose | | | | | | |
| | Colloidal silicon dioxide | 2.50 | 0.63 | 2.50 | 0.63 | 2.50 | 0.63 |
| | Magnesium Stearate | 2.50 | 0.63 | 2.50 | 0.63 | 2.50 | 0.63 |
| | Theoretical end weight | 400.00 | 100.00 | 400.00 | 100.00 | 400.00 | 100.00 |

@Palbociclib free base
* The granulation/dispersion solvent removed during process

For Preparation of 150 Capsules:
(a) Part I—In 18.75 g solution of Palbociclib free base, obtained in Example 2 (in methanol/dichloromethane) was added 37.5 g of starch in vessel, solvent was partially removed in the same vessel, then the residue was dried, followed by vacuum drying, the dried blend of Palbociclib with at least one of hydrophilic excipients was used for milling by using a jet mill to get co-milled Palbociclib with particle size less than about 20 microns as determined by microscope.
(b) Part II—povidone was then dissolved in ethanol to obtain a granulation solution or, step (b) granulate by using ethanol or purified water or a hydroalcoholic solvent.
(c) Granules were dried at 50° C. to 60° C. The dried granules were then sieved through a 30 mesh screen.
(d) Dried granules were mixed with extra granular excipients and filled in to capsules.

### Example 6 to 7

Table 4 shows the formulation of Palbociclib by a dry granulation process:

TABLE 4

| | | Examples | | | |
|---|---|---|---|---|---|
| | | 6 | | 7 | |
| | Ingredient | mg/cap | % | mg/cap | % |
| Part I | Palbociclib free base | Eq. to | 31.25 | Eq. to | 31.25 |
| (Dispersion, | solution (in Methanol/ | 125.00@ | | 125.00@ | |
| dring, | Dichloromethane) | | | | |
| Co-Milling) | from Example 2 | | | | |
| | Starch 1500 | 250.00 | 62.50 | NA | NA |
| | Lactose Monohydrate | NA | NA | 250.00 | 62.50 |
| Part II | Sodium starch Glycolate | 5.00 | 1.25 | 5.00 | 1.25 |
| (Pre Mix for | | | | | |
| Slugging) | | | | | |
| Extra granular | Microcrystalline cellulose | 15.00 | 3.75 | 15.00 | 3.75 |
| | Colloidal silicon dioxide | 2.50 | 0.63 | 2.50 | 0.63 |
| | Magnesium Stearate | 2.50 | 0.63 | 2.50 | 0.63 |
| | Theoretical end weight | 400.00 | 100.00 | 400.00 | 100.00 |

@Palbociclib free base
*The dispersion solvent removed during process

For Preparation of 150 Capsules:
(a) Part I—In a solution of Palbociclib free base (18.75 g) obtained in Example 2 (in the solution of methanol/dichloromethane) was added 37.50 g of starch in a vessel, the solvent was partially removed in the same vessel and then dried, followed by vacuum drying, and the dried blend of Palbociclib with at least one of hydrophilic excipient was used for milling by using a jet mill to get co-milled Palbociclib with particle size less than about 20 microns as determined by microscope observation.
(b) Part II—Premix for Slugging: The co-milled blend of above step (a) Part-I, was mixed in a blender with a disintegrant, i.e., sodium starch glycolate for 5 minutes;
(c) The blend of above step (b) was pressed into slugs and then milled by using multimill with 2.5 followed by 1.5 mm screen, and all granules passed through 20 mesh screen.
(d) Granules from step (c) were mixed with extragranular excipient and filled in capsules.

The dissolution rate was improved by co-milling with Palbociclib with Starch/Palbociclib with Lactose, as compared with where Palbociclib was milled alone.

1. A Palbociclib composition comprising Palbociclib co-milled with at least one hydrophilic excipient.

2. The Palbociclib composition of claim 1, wherein about 85% or more of the composition can be dissolved in 30 minutes in 900 ml of 0.1N HCl.

3. The Palbociclib composition of claim 1, wherein the hydrophilic excipient is a monosaccharide, polysaccharide, or disaccharide, or a combination thereof.

4. The plabociclib composition of claim 1, wherein the at least one hydrophilic excipient is starch.

5. The plabociclib composition of claim 1, wherein the hydrophilic excipient is pregelatinized starch.

6. The plabociclib composition of claim 1, wherein the hydrophilic excipient is selected from lacrose, starch, calcium carbonate, cellulose, mannitol, sorbitol, povidone, silicic acid, beta cyclodextrin, polyethylene glycol, and combinations thereof.

7. The Palbociclib composition of claim 1, wherein the Palbociclib: hydrophilic excipient weight ratio is about 1:10 to 10:1.

8. The Palbociclib composition of claim 1, wherein the Palbociclib: hydrophilic excipient weight ratio is about 1:5 to 5:1.

9. The Palbociclib composition of claim 1, wherein the Palbociclib: hydrophilic excipient weight ratio is about 1:3 to 1:1.

10. The Palbociclib composition of claim 1, wherein the Palbociclib: hydrophilic excipient weight ratio is about 1:2.

11. The Palbociclib composition of claim 1, wherein the composition has a d(10) of about 50 μm or less, d(50) of about 200 μm or less, d(90) of about 400 μm or less.

12. The Palbociclib composition of claim 1, wherein the composition has a d(10) of about 25 μm or less, d(50) of about 100 μm or less, d(90) of about 200 μm or less.

13. The Palbociclib composition of claim 1, wherein the composition has a d(10) of about 15 μm or less, d(50) of about 50 μm or less, d(90) of about 100 μm or less.

14. The Palbociclib composition of claim 1, wherein the composition has a preferably particle size is d(10) of 1 to 5 μm, d(50) 5 to 10 μm and d(90) less than 20 μm.

15. The Palbociclib composition of claim 1, further comprising one or more of a pharmaceutical acceptable excipient selected from a binder, filler, disintegrant, and lubricant.

16. The Palbociclib composition of claim 1, further comprising one or more pharmaceutical excipients selected from povidone, microcrystalline cellulose, sodium starch glycolate, croscarmellose sodium, and magnesium stearate.

17. The Palbociclib composition of claim 1 in the form of granules or powder.

18. The Palbociclib composition of claim 1, further comprising an organic solvent to prepare Palbociclib dispersion, wherein the organic solvent is selected from methanol, ethanol, isopropyl alcohol, ethyl acetone, dichloromethane, tetrahydrofuran and mixtures thereof.

19. The Palbociclib composition of claim 1, having a powder X-ray diffraction pattern comprising peaks at different angles (2Θ) of 8.0±0.2, 10.1±0.2, 10.3±0.2 and 11.5±0.2.

20. A method for preparing Palbociclib composition comprising:
1) preparing a Palbociclib free base solution: (a) dissolving Palbociclib dihydrochloride in water, (b) adding the solution obtained in step (a) dropwise to a mixture of ammonium hydroxide, methanol, and dichloromethane, and (c) separating layers to collect lower layer for further process;
2) adding a hydrophilic excipient to the free base Palbociclib solution obtained from step 1 (c);
3) partially removing the solvent from the solution of step 2, then drying followed by vacuum drying to obtain a dried blend of Palbociclib with at least one hydrophilic excipient;
4) milling the dried mixture from step (3) through at least one of a jet mill, rolling mill, hammer mill, centrifugal-impact mill and sieve, pebble mill, cutter mill, runner mill, or mortor and pestle; and
5) optional milling the Palbociclib mixture to an estimated size of d(10) of 1 to 5 μm, d(50) 5 to 10 μm, and d(90) less than 20 μm.

21. A method for preparing a Palbociclib composition, comprising co-milling Palbociclib and at least one hydrophilic excipient to form the Palbociclib composition.

22. The method of claim 21, wherein about 85% or more of the composition is soluble in 30 minutes in 900 ml 0.1 N HCl.

23. The method of claim 21, wherein the co-milling is performed using at least one of a jet mill, rolling mill, hammer mill, centrifugal-impact mill and sieve, pebble mill, cutter mill, runner mill, or mortor and pestle.

24. The method of claim 21, wherein the hydrophilic excipient is a monosaccharide, Polysaccharide, Disaccharide, or a mixture thereof.

25. The method of claim 21, wherein the Palbociclib: hydrophilic excipient weight ratio is about 1:10 to 10:1.

26. The method of claim 21, wherein the co-milled Palbociclib has a d(10) of 1 to 5 μm, d(50) 5 to 10 μm, and d(90) less than 20 μm.

27. The method of claim 21, further comprising wet granulation or dry granulation process.

28. The method of claim 21, further comprising the steps of wet granulation (a) Palbociclib co-milled with hydrophilic excipient, by performing milling process of claim 23, (b) wet granulation, (c) drying, (d) sizing and sifting, and (e extra granular excipient mixing, and (f) encapsulation.

29. The method of claim 28, wherein the wet granulation comprises co-milled Palbociclib with hydrophilic excipient granulate by (a) povidone dissolve in ethanol or, granulate by using ethanol or purified water or hydroalcoholic solvent, (b) drying granules at 50° C. to 60° C. and The passing the dried granules through 30 mesh sieve screen, (c) mixing extra granular excipient with dried granules, (d) blending the mixture from step (c) for use in an encapsulation process.

30. The method of claim 21, further comprising dry granulation (a) co-milled Palbociclib with hydrophilic excipient, by performing milling process as claim 23, (b) dry granulation—slugging and deslugging, (c) extra granular excipient addition, and (d) encapsulation.

31. The method of claim 30 further comprising the steps of
(a) preparing a slug by using co-milled Palbociclib of claim 20,
(b) for sizing slug use multimill with 2.5 mm followed by 1.5 mm screen, until all granules pass through 20 mesh screen,
(c) extra granular excipients mix with step (b) granules, and
(d) using the step (C) blend for encapsulation process.

32. The method of claim 21, further comprising mixing co-milled Palbociclib with an extragranular disintegrant selected from carboxymethylcellulose sodium, Sodium starch Glycolate, Collidal silicon dioxide, crospovidone, microcrystalline cellulose, and starch.

33. The method of claim 21, further comprising mixing co-milled Palbociclib with an extragranular glidant selected from colloidal silicon dioxide, magnesium trisilicate, powdered cellulose, starch, and talc.

34. The method of claim 21, further comprising mixing co-milled Palbociclib with an extragranular lubricant selected such as magnesium stearate, hydrogenated castrol oil, mineral oil, polyethylene glycol, sodium steryl fumarate, and sodium lauryl sulfate.

35. The method of claim 21, further comprising encapsulation by using capsules, such as hard gelatin capsule or hydroxypropyl methyl cellulose based capsules.

36. The method of claim 21, further comprising encapsulation by using hard gelatin capsule.

37. A method of treating cancer, comprising administering to a subject in need of such treatment a therapeutically effective amount of a pharmaceutical composition of any one of claims 1 to 19.

38. The method of claim 36, wherein the cancer is breast cancer.
